(19) United States
(12) Patent Application Publication
(10) Pub. No.: US 2025/0332439 A1
(43) Pub. Date: Oct. 30, 2025

(54) Device for Treating a Tissue Portion of a Human or Animal

(71) Applicants: Thomas Klose, Koblenz (DE); Liviu Steier, Needham, MA (US)

(72) Inventors: Thomas Klose, Koblenz (DE); Liviu Steier, Needham, MA (US)

(21) Appl. No.: 18/867,417

(22) PCT Filed: Jul. 17, 2023
(86) PCT No.: PCT/EP2023/069832

§ 371 Date: Nov. 19, 2024

(30) Foreign Application Priority Data
Jul. 27, 2022 (DE) 10 2022 118 867.1

(51) Int. Cl. A61N 5/06; A61K 31/12; A61K 41/00; A61M 1/00; A61M 3/02; A61M 11/00; A61M 16/06; A61M 37/00; A61N 5/067

(52) U.S. Cl. CPC A61N 5/062; A61K 31/12; A61K 41/0061; A61K 41/008; A61M 1/84; A61M 3/0254; A61M 11/00; A61M 16/06; A61M 37/0092; A61N 5/067; A61M 2037/0007; A61M 2205/051; A61M 2205/058; A61M 2205/128; A61N 2005/0644; A61N 2005/0663

(57) ABSTRACT

The invention relates to a device (100) for treating a tissue portion (W). The device (100) comprises an application apparatus (130) for applying a first composition, a second composition and a third composition for treating the tissue portion (W) to the tissue portion (W), with the application apparatus (130) comprising at least one application opening (131) for transferring the compositions from the application apparatus (130) to the tissue portion (W). The device comprises an exposure apparatus (140) for exposing the compositions applied to the tissue portion (W) to light, with the exposure apparatus (140) comprising an exit area (141) for the exit of the light from the exposure apparatus (140). The device (100) comprises a carrying apparatus (150), with the carrying apparatus (150) carrying the at least one application opening (131) and the exit area (141). The application apparatus (130) is designed to accommodate a replaceable cartridge (200) containing the first composition, the second composition and/or the third composition.

### TECHNICAL FIELD

The invention relates to a device for treating a tissue section, in particular a wound, of a human or an animal, the device comprising an application device for applying a first composition, a second composition and a third composition for treating the tissue section on the tissue section and an exposure device for exposing the compositions applied to the tissue section with light and a carrying device, wherein the carrying device carries the at least one application opening and the exit surface.

### STATE OF THE ART

Photodynamic therapy (PDT) is understood to mean a method for the treatment, inter alia, of tumors and other tissue changes, such as, for example, vessel developments with light in combination with a light-activatable substance, a so-called photosensitizer, and oxygen present in the tissue. For this purpose, such a primary non-toxic sensitizer or one of its metabolic precursors is administered to the patient either systemically or locally, which accumulates selectively in the tumor, or the tissue change due to certain properties of the tumor or the tissue change. After a certain waiting time, the tumor or the tissue change is irradiated with light of suitable wavelength. In this case, toxic substances, above all reactive oxygen species, are produced by photophysical processes, that damage the tumor or tissue change.

Photodynamic disinfection, also known as antibacterial photodynamic therapy (aPDT) or photoactivated disinfection is a method used inter alia in dentistry for the supportive treatment of bacterial infections in periodontitis (Clinical Oral Investigation (2022) 26: 3005-3010, https://doi.org/10.1007/s00784-021-04282-z; Der Hautarzt (2005) 11: 1048-1055, https://doi.org/10.1007/s00105-005-0977-Z; Der Hautarzt (2021) 1: 27-33, https://doi.org/10.1007/S00105-020-04737-6). As in photodynamic therapy, light is used in combination with a light-activatable substance, a so-called photosensitizer and existing atmospheric oxygen.

Usually, the photodynamic disinfection comprises the following steps:
a. staining of the microorganisms with a photosensitizer,
b. allowing the photosensitizer to act during an exposure time which is dependent on the indication and severity of the disease,
c. rinsing and removing the excess photosensitizer,
d. exposing the photosensitizer, with a wavelength, intensity, and exposure duration dependent on indication and severity of the disease.

The individual steps are carried out with different devices, and possibly the steps must also be carried out with different devices depending on the indication, for example, to allow exposure to different wavelengths. Furthermore, additional devices are generally necessary for further steps of wound treatment, for example for stimulating wound healing. Due to the large number of devices used, the treatment is complex and error-prone, in particular it is difficult to carry out all treatment steps exactly at the site of the wound in the case of a locally limited wound.

Moreover, known methods of photodynamic disinfection do not always lead to complete disinfection, so that an additional treatment with other disinfection methods is necessary, which further increases the treatment effort.

### Technical Task

The object of the invention is to create versatile devices and methods for the simple and effective treatment of a tissue section, in particular a wound, of a human or an animal.

### Technical Solution

The present invention provides a device according to claim 1 which solves the technical problem. Advantageous embodiments are the subject matter of the dependent claims.

The device is designed to treat a tissue section, in particular a wound, of a human or an animal. The tissue section can, for example, be a skin section or a mucosal section, in particular a section of an oral mucosa. The wound may be an external or internal tissue injury, for example, a tissue injury in an oral cavity, of a human or an animal. The tissue injury can be caused, for example, by a surgical intervention.

The device comprises an application device for applying a first composition, a second composition and a third composition for treating the tissue section on the tissue section, wherein the application device comprises at least one application opening for dispensing the compositions from the application device to the tissue section.

The device comprises an exposure device for exposing the compositions applied to the tissue section with light, wherein the exposure device comprises an exit surface for exiting the light from the exposure device.

The device comprises a carrying device, wherein the carrying device carries the at least one application opening and the exit surface. Further parts of the application device and/or of the exposure device can be arranged separately from the carrying device, so that the carrying device can be as small and light as possible. The parts arranged outside the carrying device can be connected by a number of lines, for example for conducting the compositions and/or the light, to the carrying device. The lines are preferably flexible, so that they do not impede a treatment person treating the tissue section and/or the accessibility of the tissue section.

### Advantageous Effects

Due to the fact that the at least one application opening, and the exit surface are carried by the carrying device, the application and the exposure can be carried out with the same carrying device. Consequently, the treatment of the tissue section can be carried out particularly easily and locally particularly precisely.

In particular, the carrying device prevents the compositions from being applied outside the treatment region. This requires a particularly small amount of the compositions and possible side effects are avoided. A contact of composition, in particular according to embodiments of the invention and/or containing hypochlorous acid, with mucous membranes or open wounds can lead, for example, to irritation.

In addition, the at least one application opening, and the outlet surface being carried by the same carrying device defines a defined positioning and alignment of the exit surface relative to the at least one application opening. This makes it possible to ensure that the exposure takes place precisely where the compositions were applied. As a result, a particularly efficient treatment is possible, and side effects due to an exposure of tissue outside an application area of the compositions are avoided.

Since the application device is designed for applying a first composition, a second composition and a third composition for treating the tissue section onto the tissue section with the same device, up to three different compositions can be used, in particular at the same time. Consequently, the efficacy of the treatment and the versatility of the device are increased without increasing the effort for the treatment.

### DESCRIPTION OF THE EMBODIMENTS

The device preferably comprises a rinsing device for rinsing the tissue section with a rinsing fluid, wherein the rinsing device has at least one rinsing opening carried by the carrying device for dispensing the rinsing fluid from the rinsing device to the tissue section. The at least one application opening can be identical to the at least one rinsing opening.

The device preferably comprises a suction device for extracting the rinsing fluid from the tissue section, wherein the suction device comprises at least one suction opening carried by the carrying device for receiving the rinsing fluid from the tissue section.

Further parts of the rinsing device and/or of the suction device apart from the at least one rinsing opening and/or the at least one suction opening can be arranged separately from the carrying device, so that the carrying device can be as small and light as possible. The parts arranged outside the carrying device can be connected to the carrying device by a number of lines, for example for conducting the rinsing fluid. The lines are preferably flexible so that they do not impede a treatment person treating the tissue section.

Due to the fact that the at least one rinsing opening and/or the at least one suction opening is/are also carried by the carrying device, the rinsing and/or the suction can also be carried out with the carrying device, so that the treatment can be used in a particularly simple manner and the device can be used in a particularly versatile manner. In addition, the carrying device defines the position and orientation of the rinsing opening and/or the suction opening relative to the application opening, so that the rinsing and/or the extraction can be carried out exactly at the location of the application of the compositions, so that the treatment takes place particularly accurately and efficiently.

A distance of the rinsing opening from the suction opening is preferably from 1 mm to 10 mm, preferably from 1 mm to 8 mm, particularly preferably from 1 mm to 5 mm. The mentioned distances allow a particularly efficient rinsing of the tissue section.

The application device preferably comprises a flow control unit for controlling, in particular independently of each other, a flow of each of the three compositions onto the tissue section. With the aid of the flow control unit, an optimum amount of the compositions can be applied to the tissue section in a simple and reliable manner.

The flow control unit preferably comprises a valve, particularly preferably a check valve, a pump and/or a pressure generator, particularly preferably with a compressed gas cartridge. The check valve can advantageously prevent foreign substances from entering the application device from the tissue section and contaminating the compositions. The pump and the pressure generator can support a transport of the compositions onto the tissue section and thus simplify their application.

The application device preferably comprises an atomizing unit for atomizing the first composition, the second composition and/or the third composition during application to the tissue section. By atomization, the effectiveness of the atomized composition can be increased by an enlarged surface of the composition. Thus, for example, an increased disinfection effect was found for an aqueous solution of hypochlorous acid (BOECKER, D, et al.: Safe, Effective, and cost-efficient air cleaning for popular rooms and entire build based on the disinfecting power of vaporized hypochlorous acid. International Journal of Health and Medical Engineering, 2022, year 16, no 12, p 209-213). The atomizing unit can be designed, for example, as described in U.S. Pat. No. 9,573,148 B2.

The application device is preferably designed to receive a replaceable cartridge containing the first composition, the second composition and/or the third composition. With the aid of the cartridge, the compositions can be introduced into the application device, refilled, and exchanged for other compositions particularly easily. As a result, the device can be used in a particularly simple and versatile manner.

The application device preferably comprises at least one cannula, particularly preferably one cannula for each of the compositions, for removing the compositions from the cartridge. With the cannula, the cartridge can be automatically pierced when the cartridge is inserted into the application device. Thus, the cartridge can be opened particularly simply and without risk of contaminating its contents. If a cannula is provided for each composition, mixing of the compositions which could impair their effectiveness is prevented.

The application device is preferably designed to receive a plurality of cartridges, wherein the application device preferably comprises a selection element, preferably a carousel, for selecting one of the cartridges for applying the compositions contained in the selected cartridge. As a result, different compositions for different indications can be kept ready for use in the application device and selected in a simple manner for use in disinfection.

The device may comprise a preparation device for preparing at least one of the compositions. The preparation device can be designed, for example, for the preparation of an aqueous solution of hydrogen peroxide and/or for the preparation of an aqueous solution of hypochlorous acid. The preparation device can comprise, for example, an electrolyzer. The preparation device advantageously makes it possible to prepare compositions, which cannot be stored or can be stored only at high cost, directly prior to their application.

The device preferably comprises a sound generator, preferably an ultrasonic generator, for exposing the first composition, the second composition and/or the third composition during the application to the tissue section and/or the rinsing fluid during the rinsing of the tissue section to sound, preferably to ultrasound. As a result of the exposure to sound, in particular to ultrasound, the compositions and/or the rinsing fluid can penetrate more quickly and deeper into the tissue section and cause a particularly efficient and effective rinsing and/or treatment.

The exposure device preferably comprises at least one laser, preferably at least one diode laser, for generating the light. A laser has the advantage that it provides a bundled light beam with high intensity and a defined wavelength. A diode laser is distinguished by a small size, a low mass, and a low energy requirement. The diode laser can be integrated, for example, into the carrying device. The exposure device can comprise at least one incandescent lamp, in particular at least one halogen incandescent lamp, or at least one light-emitting diode (LED) for generating the light.

The exposure device is preferably designed for exposure to light having a, preferably adjustable, wavelength in a range from 150 nm to 1400 nm, preferably from 400 nm to 500 nm and/or from 800 nm to 1400 nm, particularly preferably of 405 nm, 430 nm, 470 nm and/or 490 nm. The ranges and wavelengths mentioned lead, in particular with hydrogen peroxide, curcumin, rose bengal and/or hypochlorous acid in the compositions, to a particularly effective treatment. Light having a wavelength in the range from 150 nm to 600 nm can preferably be used for photodynamic disinfection. Light having a wavelength in the range from 800 nm to 1400 nm can preferably be used to stimulate wound healing.

The exposure device is preferably designed for exposure to light having a, preferably adjustable, intensity in a range from 5 mW/cm² to 500 mW/cm², preferably from 50 mW/cm² to 150 mW/cm². The ranges mentioned lead, in particular with hydrogen peroxide, curcumin, rose bengal and/or hypochlorous acid in the compositions, to a particularly effective treatment, without damaging tissue adjacent to the tissue section.

A power of the light is, for example, from 10 mW to 500 mW, in particular from 50 mW to 150 mW, preferably 100 mW. With the stated powers, the aforementioned advantageous intensities can be achieved on an exposure area of the tissue section on the order of 1 cm² suitable for an efficient and precise treatment.

The exposure device is preferably designed for exposure to light having a preprogrammed sequence of different wavelengths. By means of a preprogrammed sequence, different absorption ranges of one or more photosensitizers can be activated in an automated manner in order to achieve a particularly effective treatment, in particular against a plurality of different microorganisms. In an exemplary sequence, which in particular shows a particularly effective disinfection with hydrogen peroxide, rose bengal, curcumin and/or hypochlorous acid, exposures are carried out successively with light having wavelengths of 405 nm, 430 nm, 470 nm and 490 nm for 10 minutes each.

The exposure device is preferably designed for exposure to light during a pre-programmed period of time, for example from 1 min to 20 min, preferably from 2 min to 15 min, particularly preferably from 3 min to 10 min. A pre-programmed period of time increases the safety of the treatment, since an excessively short exposure, which could lead to incomplete disinfection, and an excessively long exposure, which could damage tissue adjacent to the tissue section, can be avoided. The mentioned periods of time lead, in particular with hydrogen peroxide, rose bengal, curcumin and/or hypochlorous acid, to an effective and safe disinfection.

The rinsing device is preferably designed for rinsing the tissue section with a gaseous rinsing fluid, preferably with air, and/or with a liquid rinsing fluid, preferably with an aqueous solution. The aqueous solution can be a, preferably physiological, saline solution.

The rinsing device preferably comprises a pressure generator, for example a pump, for supplying the rinsing fluid with a preferably adjustable overpressure in a range from 1 bar to 80 bar above an atmospheric pressure. With an overpressure in the said region, the tissue section can be effectively rinsed, quickly and for adjacent tissue in a gentle manner. An adjustable overpressure has the advantage that it can be adapted to the type of infection and/or of the tissue adjacent to the tissue section.

The rinsing device preferably comprises a reservoir containing the rinsing fluid. The device can thus advantageously be operated independently of an external source for the rinsing fluid.

The rinsing device preferably comprises a connection device for connecting the rinsing device to an external supply line for supplying the rinsing device with the rinsing fluid. As a result, the rinsing device does not require a reservoir for the rinsing fluid and can therefore be constructed in a particularly space-saving manner.

The suction device preferably comprises a pump for extracting the rinsing fluid with a, preferably adjustable, partial vacuum of 0.1 bar to 0.8 bar, preferably of 0.1 bar to 0.3 bar, particularly preferably of 0.1 bar to 0.2 bar, below atmospheric pressure. With a partial vacuum in the above-mentioned range, the rinsing fluid can be extracted from the tissue section effectively, quickly, and gently for adjacent tissue. An adjustable partial vacuum has the advantage that it can be adapted to the type of infection and/or of the tissue adjacent to the tissue section.

The suction device preferably comprises a disposal vessel for receiving the extracted rinsing fluid. The device can thus advantageously be operated independently of an external sink for the rinsing fluid.

The suction device preferably comprises a connection device for connecting the suction device to an external disposal line for disposing of the extracted rinsing fluid. As a result, the suction device does not require a disposal vessel for the rinsing fluid and can therefore be constructed in a particularly space-saving manner.

The device preferably comprises a, preferably programmable, control device for, preferably automated, control of the rinsing device, of the suction device, of the application device and/or of the exposure device. The control device can cause the device to automatically carry out the individual steps of the treatment of the tissue section with parameters adapted to the type of infection and/or the tissue adjoining the tissue section, so that the treatment can be carried out more easily and safely. A manual adjustment of individual, several, or all parameters before or during the treatment can be possible.

The control device comprises, for example, a computing device communicatively connected to the rinsing device, to the suction device, to the application device and/or to the exposure device. The control device preferably comprises a user interface, for example a touch screen, for interaction with the treatment person.

The carrying device is preferably designed as a handpiece to be held with a hand for carrying out the treatment. Dimensions, shape, and mass of the handpiece are preferably designed such that the treatment person can carry out the treatment with the handpiece ergonomically.

Since the application opening and the exit surface and preferably the rinsing opening and/or the suction opening are integrated into the handpiece, the treatment person can perform several or even all steps of treatment with the same handpiece. Consequently, the treatment can be carried out particularly easily and locally particularly precisely.

In particular, the integration in the handpiece prevents the compositions from being applied outside the treatment region, and the exposure from taking place outside the treatment region. This requires a particularly small amount of the compositions, and possible side effects are avoided.

The carrying device is preferably designed as a tent for receiving the human or the animal. The tent may be configured to receive the human or animal fully or partially, For example, the tent may be configured to receive a limb, two limbs, a lower body and/or an upper body. The tent can be designed, for example, like a sauna tent. The configuration of the carrying device as a tent is particularly suitable for the efficient treatment of large-area tissue sections.

In the embodiment of the carrying device as a tent, the application device can comprise, for example, a droplet irrigation, an evaporation device and/or a nebulizing device for the compositions in the tent. As a result, the compositions can also be applied efficiently to large-area tissue sections.

In the embodiment of the carrying device as a tent, the exposure device can comprise, for example, an LED strip and/or a number of light guides for the large-area exposure of the compositions.

The carrying device is preferably designed as a breathing mask for ventilating the human or animal. Breathing masks often result in infections and inflammations of skin and mucous membranes, in particular in the case of long-lasting use. If the carrying device is designed as a breathing mask, this results in the advantage that an infection or inflammation can be treated in a particularly simple manner with the device, without interrupting the respiration of the human or the animal.

The invention relates to a cartridge for use in a device according to embodiments of the invention. The application device of the device is preferably designed for the exchangeable accommodation of the cartridge. The invention also relates to a device according to embodiments of the invention comprising a cartridge according to embodiments of the invention, wherein the cartridge is preferably accommodated exchangeably by the application device of the device.

The cartridge comprises a first reservoir containing the first composition, preferably a second reservoir, which is separate from the first reservoir in a liquid-tight manner and contains the second composition, and particularly preferably a third reservoir containing the third composition separated in a liquid-tight manner from the first reservoir and from the second reservoir. This prevents mixing of the compositions which could lead to undesirable interactions which could impair the effectiveness of the treatment. Nevertheless, only a single cartridge is necessary for the compositions, so that the procurement, storage, and replacement of the cartridge is simpler than if a plurality of cartridges were necessary.

The cartridge preferably comprises a first removal opening for removing the first composition from the first reservoir, particularly preferably a second removal opening for removing the second composition from the second reservoir, and most preferably a third removal opening for removing the third composition from the third reservoir. Undesired mixing of the compositions is prevented by separate removal openings.

Preferably, at least one, in particular each, of the removal openings is closed by a valve, particularly preferably by a check valve, and/or by a septum. A check valve has the advantage that it prevents foreign matter from entering the associated reservoir. A septum has the advantages that it reliably closes the associated removal opening and can be opened in a simple manner, for example, with a cannula of the application device of the device.

Preferably, at least one, in particular each, of the reservoirs of the cartridge is sealed in a light-tight manner. Thus, it is possible to store compositions containing light-sensitive components in the reservoir without additional protective measures.

At least one, in particular each, of the compositions has an overpressure above atmospheric pressure in the cartridge. As a result, as soon as the cartridge is opened, the composition can emerge from the cartridge driven by the overpressure and be applied to the tissue section without an additional drive, for example a pump, being necessary. The device can thus be constructed in a particularly simple manner.

The first composition that can be applied by the device and/or is contained in the cartridge preferably contains curcumin, rose bengal, 5-aminolevulinic acid, embelin and/or methylene blue. These photosensitizers, in particular a combination of curcumin with rose bengal, allow particularly effective disinfection of the tissue section.

The first composition that can be applied by the device and/or is contained in the cartridge preferably contains chitosan nanoparticles. Addition of chitosan nanoparticles can enhance the effectiveness of the photosensitizers, in particular of the aforementioned photosensitizers.

The second composition that can be applied by the device and/or is contained in the cartridge contains or is preferably hypochlorous acid, preferably as an aqueous solution with a molar concentration of from 0.01% to 1%, particularly preferably from 0.04% to 0.5%. A two-stage disinfection with such a second composition and a first composition designed as described above, in particular with curcumin and rose bengal, is particularly effective.

The hypochlorous acid preferably does not contain sodium hypochlorite since it acts in a vessel-damaging manner. Hypochlorous acid is microbially unsusceptible because of its action as an oxidizing agent and because of its low pH.

The third composition which can be applied by the device and/or is contained in the cartridge contains or is, for example, an aqueous solution of chlorhexidine, in particular with a chlorhexidine concentration of from 0.05% to 0.25%, preferably from 0.1% to 0.2%, particularly preferably of 0.12%.

The third composition which can be applied by the device and/or is contained in the cartridge contains or is, for example, an aqueous solution of hydrogen peroxide, in particular with a hydrogen peroxide concentration of from 0.5% to 10%, preferably from 1% to 5%, particularly preferably from 1.5% to 3.0%. Chlorhexidine and hydrogen peroxide in the concentrations mentioned each show an effective wound disinfection which can be intensified by light exposure.

The cartridge preferably comprises a temperature indicator for indicating an exceedance of a maximum storage temperature of the cartridge and/or for indicating an undershooting of a minimum storage temperature of the cartridge. With the temperature indicator, a person using the cartridge can easily detect whether it has been stored outside the intended temperature range, as a result of which the effectiveness of the compositions could be impaired. Thus, the safety of the use of the cartridge is increased.

The temperature indicator can contain, for example, at least one dye which changes its color when the maximum storage temperature is exceeded or undershooting of the minimum storage temperature occurs.

The cartridge is preferably to be stored in the refrigerator and, in connection with its use, must not be left unsealed or exposed to light for an unnecessarily long period of time, in order to ensure the stability and freedom of germs of the compositions. The temperature effect on the stability can be strong. For example, the decomposition rate of hypochlorous acid is doubled between 20° C. and 25° C.

The invention relates to a composition for treating a tissue section, in particular a wound, of a human or an animal with a device according to embodiments of the invention. The invention also relates to a device according to embodiments of the invention and to a cartridge according to embodiments of the invention, which each comprise the composition according to embodiments of the invention as a first composition.

The composition is gel-like. It is thus a disperse system comprising a solid and a liquid component. The solid component forms a sponge-like, three-dimensional matrix, the pores of which are filled by a liquid component. The liquid component is thereby immobilized in the solid component. Thus, in contrast to a liquid composition, the gel-like composition remains there where it is applied to the tissue section. As a result, a particularly small amount of the composition is required, and possible side effects due to an uncontrolled spreading of the composition are avoided. Contact of a composition, in particular a composition according to embodiments of the invention, with mucous membranes or open wounds could lead, for example, to irritation.

The composition comprises curcumin with a mass fraction of 0.1% to 10%, preferably of 0.5% to 5%, of the composition. The gel-like composition comprises rose bengal with a mass fraction of 0.01% to 1%, preferably of 0.05% to 0.5%, of the composition. The combination of curcumin and rose bengal in the stated proportions leads to a particularly effective disinfection of the tissue section.

The composition comprises a gel-forming composition and water. The gel-forming composition forms the solid matrix of the gel-like composition. Water serves as a carrier substance which is acceptable for wound treatment for the remaining components of the gel-like composition.

The gel-forming composition preferably contains a poloxamer, for example poloxamer 407, with a mass fraction of 0.2% to 20%, preferably of 1% to 10%, of the composition. Poloxamers are block copolymers of ethylene oxide and propylene oxide. These are low-foaming and foam-damping, nonionic surfactants. Poloxamer 407 has a relative mass fraction of the ethylene oxide units of 70%, a relative molecular weight of the polypropylene glycol block of 4000 and an entire average molecular weight of 9840 to 14600.

The gel-forming composition preferably contains 1,2-propylene glycol with a mass fraction of 0.13% to 13%, preferably from 0.63% to 6.3%, of the composition. 1,2-propylene glycol, also known as 1,2-propanediol, is a clear, colorless, virtually odorless, and strongly hygroscopic liquid. 1,2-Propylene glycol is a health-acceptable solvent and has antimicrobial properties, so that additional preservatives can be dispensed with.

The gel-forming composition preferably contains 2-propanol with a mass fraction of 0.13% to 13%, preferably from 0.63% to 6.3%, of the composition. 2-Propanol (IUPAC-name propan-2-ol), also known as isopropyl alcohol or isopropanol (abbreviated IPA), is the simplest non-cyclic, secondary alcohol and a monohydric alcohol. 2-Propanol is a solvent with low toxicity and has antimicrobial properties, so that additional preservatives can be dispensed with.

The gel-forming composition preferably contains medium chain-length triglycerides having a mass fraction of from 0.05% to 5%, preferably from 0.25% to 2.5%, of the composition. Triglycerides, also called glycerol triesters, are triple esters of the trivalent alcohol glycerol with three fatty acid molecules and should be referred to as triacylglycerols according to the IUPAC recommendation. Medium chain-length triglycerides have medium length fatty acids (6 to 12 C atoms).

The composition is durable from its production preferably for at least 6 months, particularly preferably for at least 12 months, in particular when the composition is stored at refrigerator temperature.

The invention relates to a method for producing a composition according to embodiments of the invention.

The method comprises mixing the curcumin, the rose bengal, the poloxamer, the 1,2-propylene glycol, the 2-propanol and the water to form a mixture. The mixing takes place, for example, with a plunger or with a pestle in a mortar, in particular in a catch tray.

The method comprises leaving the mixture for a period of rest of at least 30 min, preferably at least 45 min, particularly preferably at least 60 min. The resting results in air bubbles and/or gel lumps formed during mixing being able to dissolve.

During resting, the mixture is preferably covered to avoid contamination, and may occasionally be stirred to aid in dissolution of gel clumps and/or air bubbles.

The process comprises mixing the medium chain-length triglycerides into the mixture after resting. Mixing results in a homogeneous gel.

Evaporation losses occurring during mixing, resting and/or mixing can be compensated for by stirring 2-propanol into the composition.

The method may comprise adjusting a pH of the composition to a slightly acidic pH of preferably 6.0 to 6.7. A slightly acidic pH reduces the proliferation capacity of bacteria frequently encountered in wound infections, for example Propionibacterium acnes.

The method can comprise filling the composition into a vessel, in particular into a cartridge according to embodiments of the invention, wherein the vessel is tightly sealed after filling in order to avoid contamination.

The composition is preferably stored at refrigerator temperature, for example at a temperature of 3° C. to 9° C. in order to reduce the growth of microorganisms.

### EXAMPLES

The composition according to an embodiment of the invention consists, for example, of the following ingredients per 100 g of the composition:
a. 10.0 g curcumin,
b. 1.0 g rose bengal,
c. 20.0 g poloxamer 407,
d. 12.5 g propylene glycol,
e. 12.5 g 2-propanol,
f. 5.0 g medium chain-length triglycerides and
g. 39.0 g water.

The method according to an embodiment of the invention for producing a composition according to an embodiment of the invention, for example from the ingredients mentioned in the preceding paragraph, comprises, for example, the following steps:
a. Curcumin, rose bengal, poloxamer, propylene glycol, purified water and 2-propanol are carefully mixed in a catch tray tared with a pestle. Air bubbles and gel clumps in the mixture may occur in this manufacturing step.
b. The mixture is allowed to rest for at least 60 min with occasional stirring. The mixture is clear and viscous after 60 min and shows few air bubbles.
c. The medium chain-length triglycerides are carefully mixed into the mixture. The mixture gets white and viscous and shows a few air bubbles.
d. Evaporation losses are compensated for with 2-propanol and stirred.
e. The finished gel-like composition must look white and homogeneous and smell like 2-propanol and must not contain any air bubbles.
f. The pH should be 6.0 to 6.7. Since bacteria, for example Propionibacterium acnes, multiply a pH of at least 6.8, the pH is optionally adjusted to below 6.8.
g. The gel-like composition is directly filled and tightly sealed.

A treatment of a wound carried out with the device according to embodiments of the invention comprises, for example, the following steps, which are repeated, for example, twice per week for four weeks:
a. rinsing the wound with the rinsing fluid to remove damaged tissue and/or foreign bodies,
b. applying the first composition, in particular the composition according to embodiments of the invention, for example as a thin film having a surface density of from 0.05 g/cm² to 0.2 g/cm², into the wound,
c. exposing the first composition at least with wavelengths of 405 nm, 430 nm, 470 nm, and 490 nm for 10 min each,
d. wiping the first composition after exposure, for example with a mild facial water,
e. applying the second composition, in particular an aqueous solution of hypochlorous acid with a molar concentration of 400 ppm, where optionally reactive oxygen species can be removed with citric acid,
f. exposing the second composition at least with wavelengths of 405 nm, 430 nm, 470 nm, and 490 nm for 10 min each

A further treatment of a wound performed with the device according to embodiments of the invention comprises, for example, the following steps:
a. applying a composition, in particular an aqueous solution of hydrogen peroxide with a mass fraction of 1.5% or 3.0%, or an aqueous solution of chlorhexidine with a mass fraction of 0.12%, into the wound,
b. exposing the composition to light having a wavelength of 405 nm during an exposure period of 3 min.

### BRIEF DESCRIPTION OF THE DRAWINGS

Further advantages, objects and properties of the invention will be explained with reference to the following description and accompanying drawings, in which embodiments of the invention are illustrated by way of example.

FIG. 1 shows a schematic illustration of a device according to an embodiment of the invention.

FIG. 2 shows a schematic representation of a cartridge according to an embodiment of the invention.

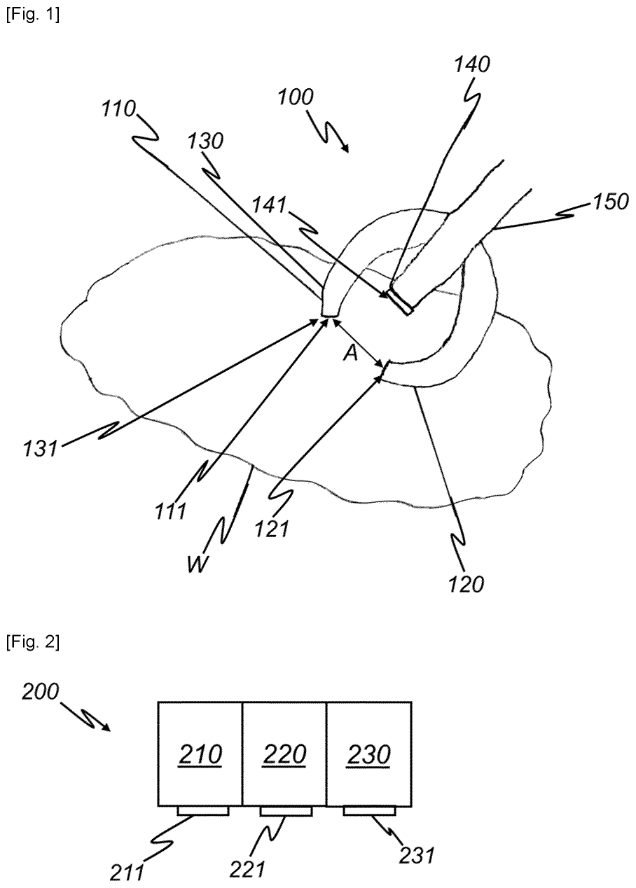

### FIG. 1

FIG. 1 shows a schematic illustration of a device 100 according to an embodiment of the invention for treating a tissue section W, in particular a wound, of a human or an animal.

The device 100 shown comprises a rinsing device 110 for rinsing the tissue section W with a rinsing fluid, wherein the rinsing device 110 comprises a rinsing opening 111 for dispensing the rinsing fluid from the rinsing device 110 to the tissue section W.

The device 100 shown comprises a suction device 120 for extracting the rinsing fluid from the tissue section W, wherein the suction device 120 comprises a suction opening 121 for receiving the rinsing fluid from the tissue section W into the suction device 120.

The device 100 shown comprises an application device 130 for applying a first composition, a second composition and a third composition for treating the tissue section W onto the tissue section W, wherein the application device 130 comprises an application opening 131 for dispensing the compositions from the application device 130 to the tissue section W.

The application opening 131 is arranged, for example, close to the rinsing opening 111. The application opening 131 can be identical to the rinsing opening 111.

The device 100 shown comprises an exposure device 140 for exposing the compositions applied to the tissue section W with light, wherein the exposure device 140 comprises an exit surface 141 for exiting the light from the exposure device 140. The exposure device 140 comprises, for example, a diode laser for generating the light.

The device 100 shown comprises a carrying device 150 designed as a handpiece, wherein the rinsing opening 111, the suction opening 121, the application opening 131 and the outlet surface 141 are integrated into the handpiece 150.

A distance A of the rinsing opening 111 from the suction opening 121 is, for example, from 1 mm to 5 mm.

### FIG. 2

FIG. 2 shows a schematic illustration of a cartridge 200 according to an embodiment of the invention for use in a device 100 according to embodiments of the invention.

The cartridge 200 shown comprises a first reservoir 210 containing the first composition, for example a first composition according to an embodiment of the invention, and a second reservoir 220, separated from the first reservoir (210) in a liquid-tight manner, containing the second composition, for example an aqueous solution of hypochlorous acid. The cartridge 200 comprises a third reservoir 230, which is separated from the first reservoir 210 and from the second reservoir 220 in a liquid-tight manner, and contains the third composition, for example an aqueous solution of hydrogen peroxide.

The cartridge shown has a first removal opening 211 for removing the first composition from the first reservoir 210, a second removal opening 221 for removing the second composition from the second reservoir 220 and a third removal opening 231 for removing the third composition from the third reservoir 230.

### LIST OF REFERENCE NUMBERS

100 device
110 rinsing device
111 rinsing opening
120 suction device
121 suction opening
130 application device
131 application opening
140 exposure device
141 exit surface
150 carrying device
200 cartridge
210 first reservoir
211 first removal opening
220 second reservoir
221 second removal opening
230 third reservoir
231 third removal opening
A distance
W tissue section

1. A device for treating a tissue section, in particular a wound, a human or an animal, the device comprising:
a. an application device for applying a first composition, a second composition, and a third composition for treating the tissue section, wherein the application device comprises at least one application opening for dispensing the first, second, and third compositions from the application device to the tissue section;
b. an exposure device for exposing the first, second, and third compositions applied to the tissue section with light, the exposure device having an exit surface for exiting the light from the exposure device;
c. a carrying device, wherein the carrying device carries the at least one application opening and the exit surface; and
d. a sound generator for exposing the first composition, the second composition, and/or the third composition during application to the tissue section and/or rinsing fluid during the rinsing of the tissue section to sound;
wherein
the exposure device is designed for exposure to light having a wavelength in a range from 400 nm to 500 nm and in a range from 800 nm to 1400 nm,
the device comprises a rinsing device for rinsing the tissue section with the rinsing fluid, wherein the rinsing device comprises at least one rinsing opening carried by the carrying device for dispensing the rinsing fluid from the rinsing device to the tissue section,
the rinsing device comprises a pressure generator for supplying the rinsing fluid with an overpressure in a range from 1 bar to 80 bar above an atmospheric pressure,
at least one exchangeable cartridge containing at least the second composition and the third composition is accommodated by the application device,
the second composition is an aqueous solution of hypochlorous acid, and
the third composition is an aqueous solution of hydrogen peroxide.

2. The device of claim 1,
further comprising a suction device for extracting the rinsing fluid from the tissue section, wherein the suction device comprises at least one suction opening carried by the carrying device for receiving the rinsing fluid from the tissue section into the suction device.

3. The device according to claim 1,
wherein the application device comprises a flow control unit for controlling a flow of each of the three compositions onto the tissue section.

4. The device according to claim 1,
wherein the application device comprises an atomizing unit for atomizing the first composition, the second composition, and/or the third composition upon application to the tissue section.

5. (canceled)

6. The device according to claim 1,
wherein the exposure device is designed for exposure to light having an intensity in a range of 50 mW/cm² to 150 mW/cm².

7. (canceled)

8. The device according to claim 1,
wherein the carrying device is configured as a hand piece to be held by a treatment person with a hand for treating the tissue section.

9. The device according to claim 1,
wherein the carrying device is configured as a tent for receiving the human or animal.

10. The device according to claim 1,
wherein the carrying device is configured as a breathing mask for respiration of the human or animal.

11. The device according to 1, wherein the cartridge comprises
a. a first reservoir containing the first composition,
b. a second reservoir, which is separate from the first reservoir in a liquid-tight manner and contains the second composition, and
c. a third reservoir, separated from the first reservoir and from the second reservoir in a liquid-tight manner, containing the third composition.

12. The device according to claim 9,
wherein
the cartridge comprises a first removal opening for removing the first composition from the first reservoir, a second removal opening for removing the second composition from the second reservoir, and a third removal opening for removing the third composition from the third reservoir, wherein at least one of the removal openings is closed by a valve.

13. (canceled)

14. The device according to claim 1, wherein the first composition is gel-like and comprises the following components:
a. curcumin with a mass fraction of from 0.1% to 10% of the first composition,
b. rose bengal having a mass fraction of from 0.01% to 1% of the first composition,
c. a gel-forming composition, and
d. water.

15. The device of claim 14,
wherein
the gel-forming composition comprises:
a. a poloxamer having a mass fraction of from 0.2% to 20% of the first composition,
b. 1, 2-propylene glycol having a mass fraction of from 0.13% to 13% of the first composition,
c. 2-propanol having a mass fraction of from 0.13% to 13% of the first composition, and/or
d. medium chain-length triglycerides having a mass fraction of from 0.05% to 5% of the first composition.
